(19) United States
(12) Patent Application Publication
(10) Pub. No.: US 2009/0240172 A1
(43) Pub. Date: Sep. 24, 2009

(54) VESTIBULAR REHABILITATION UNIT

(75) Inventors: Nicolas FERNANDEZ TOURNIER, Montevideo (UY); Hamlet Suarez, Montevideo (UY); Alejo Suarez, Montevideo (UY); Dario Deisinger, Montevideo (UY)

Correspondence Address: SUGHRUE MION, PLLC, 2100 PENNSYLVANIA AVENUE, N.W., SUITE 800, WASHINGTON, DC, 20037, US

(73) Assignee: TRENO CORPORATION, Tortola (VG)

(21) Appl. No.: 12/478,347

(22) Filed: Jun. 4, 2009

Related U.S. Application Data
(63) Continuation-in-part of application No. 11/383,059, filed on May 12, 2006.
Application No. 11/383,059 is a continuation of application No. PCT/IB2004/003797, filed on Nov. 15, 2004.

(30) Foreign Application Priority Data
Nov. 14, 2003 (UY) 28083

(51) Int. Cl. A61B 5/11; A61M 21/00

(52) U.S. Cl. 600/595; 600/27

(57) ABSTRACT

An apparatus and method for enabling selective stimulation of oculomotor reflexes involved in retinal image stability. The apparatus enables real-time modification of auditory and visual stimuli according to the patient's head movements, and allows the generation of stimuli that integrate vestibular and visual reflexes. The use of accessories allow the modification of somatosensory stimuli to increase the selective capacity of the apparatus. The method involves generation of visual and auditory stimuli, measurement of patient response and modification of stimuli based on patient response. The apparatus and method may include a vestibular rehabilitation unit (VRU) and a remote training unit (RTU). Instructions may be transmitted from the VRU to the RTU, and information of detected responses may be transmitted from the RTU to the VRU.

The present application is a continuation-in-part of U.S. application Ser. No. 11/383,059, filed May 12, 2006, which is a continuation of PCT application No. PCT/IB2004/003797 filed Nov. 15, 2004, and which claims priority from Uruguayan Application No. 28083 filed on Nov. 14, 2003. These prior applications are incorporated herein in their entirety by reference.

### BACKGROUND OF THE INVENTION

1. Field of the Invention

The present invention generally relates to the application of computer technology (hardware and software) to the field of medicine. More specifically, the present invention relates to a vestibular rehabilitation system and method for treatment of balance disorders of distinct origin.

2. Description of the Related Art

A patient diagnosed with an episode of vestibular neuronitis experiences symptoms characterized by a prolonged crisis of vertigo, accompanied with nausea and vomiting. Once the acute episode remits, a sensation of instability of a non-specific nature persists in the patient, especially when moving or in spaces where there are many people. The sensation of instability affects the quality of life and increases the risk of falling, especially in the elderly, with all the ensuing complications, including the loss of life.

The mechanism underlying this disorder is a deficit in the vestibulo-oculomotor reflex, aftereffects of the deafferentiation of one of the balance receptors, the vestibular receptor, situated in the inner ear. The procedure to treat this deficit involves achieving a compensation of the vestibular system by training the balance apparatus through vestibular rehabilitation. In order to achieve this compensation, stimulation of the different systems that control the movement of the eyes is performed, as well as stimulation of the somatosensory receptors, the remaining vestibular receptor and the interaction between these components.

Other rehabilitation systems applying virtual reality, for example BNAVE (Medical Virtual Reality Center—University of Pittsburgh) and Balance Quest (Micromedical Technologies), are unable to perform real-time fusion of visual vestibular interactions, modifying the image on the retina according to a velocity of head movement in three axes.

Additionally, rehabilitation of the patient is traditionally performed with the VRU at a clinic but this often makes treatment difficult and inconvenient as patients might need to visit the clinic on a daily basis. Particularly for people living at a considerable distance, the elderly population, and others for whom travel is difficult, a system and method for home treatment is needed.

### SUMMARY OF THE INVENTION

The Vestibular Rehabilitation Unit (VRU) enables selective stimulation of oculomotor reflexes involved in retinal image stability. The VRU allows generation of stimuli through perceptual keys, including the fusion of visual, vestibular and somatosensory functions specifically adapted to the deficit of the patient with balance disorders. Rehabilitation is achieved after training sessions where the patient receives stimuli specifically adapted to his/her condition.

Using computer hardware and software, the Vestibular Rehabilitation Unit (VRU) enables real-time modification of stimuli according to the patient's head movements. This allows the generation of stimuli that integrate vestibular and visual reflexes. Moreover, the use of accessories that allow the modification of somatosensory stimuli increases the system's selective capacity. The universe of stimuli that can be generated by the VRU results from the composition of ocular and vestibular reflexes and somatosensory information. This enables the attending physician to accurately determine which conditions favor the occurrence of balance disorders or make them worse, and design a set of exercises aimed at the specific rehabilitation of altered capacities.

The aim of the Vestibular Rehabilitation Unit is to achieve efficient interaction among the senses by controlled generation of visual stimuli presented through virtual reality lenses, auditory stimuli that regulate the stimulation of the vestibular receptor through movements of the head captured by an accelerometer and interaction with the somatosensory stimulation through accessories, for example, but not limited to, an elastic chair and Swiss balls.

The software includes basic training programs. For each program, the Vestibular Rehabilitation Unit can select different characteristics to be associated with a person and a particular session, with the capacity to return whenever necessary to those characteristics that are set by defect.

The Vestibular Rehabilitation Unit also has a web mode that enables it to work remotely from the patient.

According to another exemplary embodiment, a remote training unit (RTU), located in a patient's home or other location remote from the VRU is used to provide on-location rehabilitation services to a patient. Instructions are transmitted from the VRU or other location to the RTU and are used to control a stimulus generating system to provide auditory and/or visual stimuli to a patient. While the stimuli are being provided, information is detected including vestibulo-ocular and vestibulo-spinal body responses of the patient. The information may be transmitted back tn the VRU or other remote location for analysis by a therapist.

### BRIEF DESCRIPTION OF THE DRAWINGS

Reference should be made to the following detailed description which should be read in conjunction with the following figures, wherein like numerals represent like parts:

FIG. 1 is a block diagram illustrating an exemplary embodiment of a Vestibular Rehabilitation Unit;

FIG. 2 is a flow chart illustrating an exemplary training process;

FIG. 3 is a block diagram illustrating another exemplary embodiment of the present invention; and

FIG. 4 is a flow chart illustrating another exemplary training process.

### DETAILED DESCRIPTION OF EXEMPLARY EMBODIMENTS OF THE INVENTION

The Vestibular Rehabilitation Unit (VRU) combines a computer, at least one software application operational on the computer, a stimulus generating system, a virtual reality visual helmet and a multidirectional elastic chair, for example, but not limited to, a set of Swiss balls. The system comes with a module for the calibration of the virtual reality visual helmet to be used by the patient. Alternately, the system may be modified for use with a screen, such as a flat screen of a monitor display or a television, in place of the virtual reality helmet/goggles. Certain modifications are noted below.

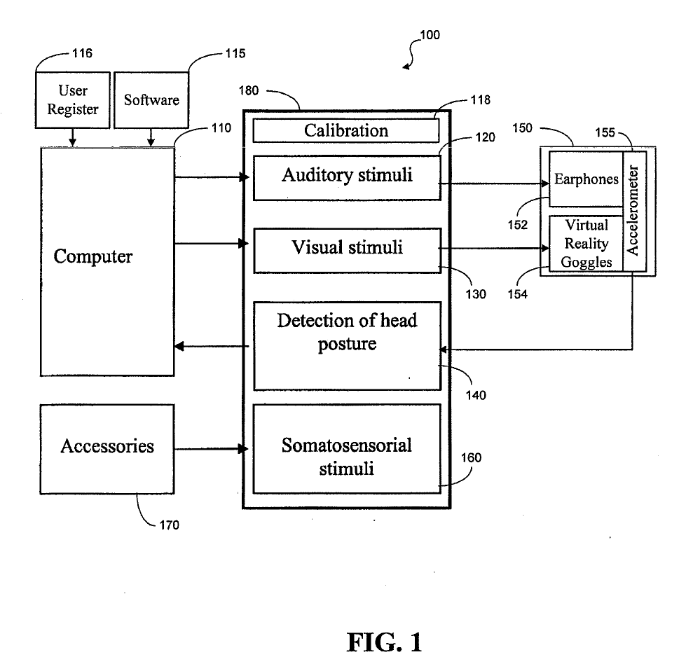

FIG. 1 is a block diagram illustrating an exemplary embodiment of a Vestibular Rehabilitation Unit.

The VRU 100 includes a computer 110, at least one software application 115 operational on the computer, a stimulus generating system 180 including a calibration module 118, an auditory stimuli module 120, a visual stimuli module 130, a head posture detection module 140, and a somatosensorial stimuli module 160, a virtual reality helmet 150, and related system accessories 170, for example, but not limited to, a mat, an elastic chair and an exercise ball. The virtual reality helmet 150 may further include virtual reality goggles 152 and earphones 154.

The computer 110, on which the VRU runs, may include a personal computer, a notebook computer, a netbook, or a game console or other apparatus having a capability of processing information and delivering visual stimulation. Any device with processing and graphics capabilities may be suitable for use as the computer 110.

The software 115 may be embodied on a computer-readable medium, for example, but not limited to, magnetic storage disks, optical disks, and semiconductor memory, or the software 115 may be programmed in the computer 110 using nonvolatile memory, for example, but not limited to, nonvolatile RAM, EPROM and EEPROM.

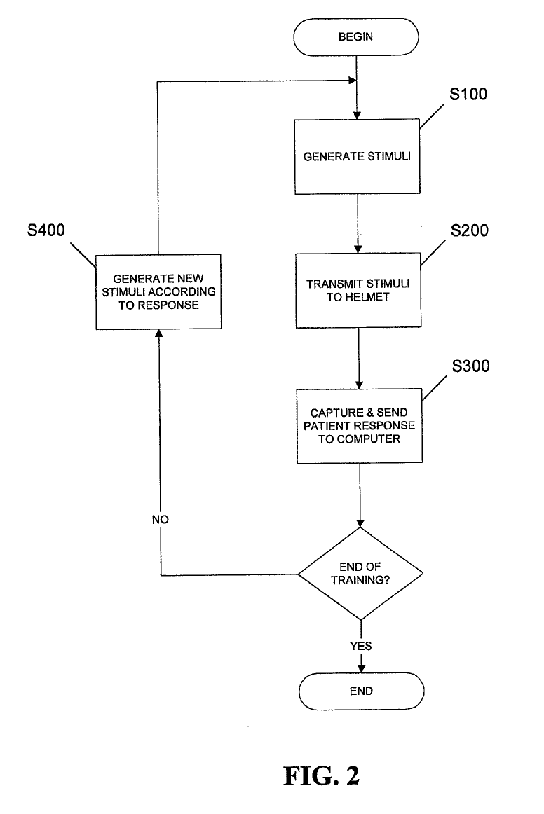

FIG. 2 is a flow chart illustrating the training process. The training process involves generating stimuli S100 by the software 115 and delivering the stimuli to the patient S200 through the virtual reality helmet 150. The response of the patient to this stimuli is captured and sent S300 by the virtual reality helmet 150 to the computer 110 where the software 115 generates new stimuli according to the detected response S400.

The software 115 generates stimuli to compensate for deficiencies detected in the balance centers of the inner ear through sounds and moving images generated in the virtual reality visual helmet 150 and interacts with the sounds and moving images to obtain more efficient stimuli. The software includes at least the following six basic training programs: sinusoidal foveal stimulus, in order to train the slow ocular tacking; random foveal stimulus in order to train the saccadic system; retinal stimulus in order to train the optokinetic reflex; visual-acoustic stimulus in order to treat the vestibular-oculomotor reflex; visual-acoustic stimulus in order to treat the visual suppression of the vestibular-oculomotor reflex; and visual-acoustic stimulus in order to the treat the vestibular-optokinetic reflex and/or the vestibular-optokinetic interaction.

For each program, the VRU 100 can select different characteristics to be associated with a person and a particular session, with the capacity to return whenever necessary to those characteristics that are set by defect. The characteristics to be determined according to a program may include: duration (in seconds); form of a figure (sphere or circle); size; color (white, blue, red or green that will be seen on a black background); direction (horizontal, vertical); mode (position on the screen, position of the edges, sense); amplitude (in degrees); and frequency (in Hertz).

Auditory and visual stimuli are delivered from the auditory stimuli module 120 and the visual stimuli module 130, respectively, to the patient wearing the virtual reality helmet 150 through the virtual reality goggles 152. The computer 100 generates visual stimuli on the displays of the virtual reality goggles 152 and auditory stimuli in the earphones 154. The implementation of auditory and visual stimuli through a virtual reality helmet 150 enables the isolation of the patient from other environmental stimuli thus achieving high specificity.

Exercises are specified for the patient during some of which the patient is asked to move the head either horizontally or vertically. The detection of the head posture is made by an accelerometer 155 (head tracker) attached to the helmet 150. The accelerometer 155 detects the head's horizontal and vertical rotation angles with respect to the resting position with the eyes looking forward horizontally. The head tracking device is not limited to an accelerometer and may include any head tracking device or method including, but not limited to, inertial devices and methods, electromagnetic devices and methods, infrared devices and methods, and ultrasound devices and methods. The head tracking device may also include a tracking system that records the movements, such as by a camera or web-cam, and algorithms to calculate head position. These head tracking methods and devices may be used in conjunction with virtual reality goggles or with a screen display.

The somatosensory stimuli are generated by the patient him/herself during exercise. The exercises may be performed using the accessories 170. These stimuli may be: stationary gait movements on a firm surface or a soft surface, for example, but not limited to, a mat; and vertical movements sitting on a ball designed for therapeutic exercise, for example, but not limited to, an elastic chair and a set of Swiss balls.

The work with the elastic chair or the Swiss balls selectively stimulates one of the parts of the inner ear involved in balance, whose function is to sense the lineal accelerations, in general gravity. In this way, when the person seated on a ball “bounces” or “rebounds,” they are stimulating the macular, utricule and/or saccule receptors and at the same time interacting with the visual stimuli generated by the software and shown through the virtual reality lenses or display screen. The movements that should be performed are specified in accordance with the visual stimulus presented, thereby training the different vestibulo-oculomotor reflexes which are of significant importance for the correct function of the system of balance.

The VRU 100 is capable of generating different stimuli for selective training of the oculomotor reflexes involved in balance function. For algorithm description purposes it is assumed that displays of the virtual reality goggles 152 cover the patient's entire visual field. Stimuli are the result of displaying easily recognizable objects. A real visual field is abstracted as a rectangle visualized by the patient in the resting position. Rx and Ry are coordinates of the center of an object in the real field.

When the patient moves his or her head, the accelerometer 155 transmits the posture-defining angles to the computer 110. An algorithm turns these angles into posture coordinates Cx and Cy on the visual field. The object is shown on the displays at Ox and Oy coordinates. The displays of the virtual reality goggles 152 accompany the patient's movements, therefore, according to the movement composition equations 1 and 2:

Rx=Cx+Ox (Equation 1)

Ry=Cy+Oy (Equation 2)

(When a display screen is used in place of virtual reality goggles Ox=Cx and Oy=Cy.) This nomenclature will be used to describe algorithms.

During the exercises involving vestibular information, the patient may be asked to move the head gently. Periodic auditory stimuli of programmable frequency are used to mark the rhythm of the movement. For example, a short tone is issued every second, asking the patient to move the head horizontally so as to match movement ends with sounds. In this case, an approximation to Cx would be Cx=k cos Πt.

Three channels are identified: the auditory channel is an output channel that paces the rhythm of the patient's movement; the image channel “0” is an output channel that corresponds to the coordinates of the object on the display; and the patient channel is an input channel that corresponds to the coordinates of the patient's head in the virtual rectangle.

The following sections involve stimuli of horizontal movements of the patient's eye. Stimuli of vertical movements of the patient's eye are similar. In the algorithms it would be enough to replace coordinate ‘x’ by the relevant ‘y’ coordinate.

In all cases a symbol, for example, a number or a letter, that changes at random is shown inside the object. The patient is asked to say aloud the name of the new symbol every time the symbol changes. This additional cognitive exercise, symbol recognition, enables the technician to check whether the patient performs the oculomotor movement. This is useful for voluntary response stimuli such as smooth pursuit eye movement, saccadic system stimulation, vestibulo-oculomotor reflex and suppression of the vestibulo-oculomotor reflex. Duration, shape, color, direction (right-left, left-right, up-down or down-up), amplitude and frequency may be programmed according to the patient's needs.

Following are stimuli that are associated to the different oculomotor reflexes. Notes regarding the use of a display screen in place of virtual reality goggles are included in parenthesis.

TABLE 1
Smooth pursuit eye movement

| | |
|---|---|
| Auditory channel | No signal |
| Patient's channel | No signal (no head movement) |
| Image channel | Ox = k cos 2 Π F t, with a programmable |
| | frequency “F”. |

The stimulus indicated in Table 1 generates a response from one of the conjugate oculomotor systems called “smooth pursuit eye movement command.” The cerebral cortex has a representation of this reflex at the level of the parietal and occipital lobes. Co-ordination of horizontal plane movements occurs at the protuberance (gaze pontine substance), and co-ordination of vertical plane movements occurs at the brain stem in the pretectal area. It has very important cerebellar afferents, and afferents from the supratentorial systems. From a functional standpoint, it acts as a velocity servosystem that allows placing on the fovea an object moving at speeds of up to 30 degrees per second. Despite the movement, the object's characteristics can be defined, as the stimulus-response latency is minimal.

This type of reflex usually shows performance deficit after the occurrence of lesions of the central nervous system caused by acute and chronic diseases, and especially as a consequence of impairment secondary to aging. The generation of this type of stimulation cancels input of information from the vestibulo-oculomotor reflex. Consequently, when there are lesions that alter the smooth pursuit of objects in the space function, training of this system stimulates improvement of its functional performance and/or stimulates the compensatory mechanisms that will favor retinal image stabilization.

TABLE 2
Saccadic system

| | |
|---|---|
| Auditory channel | No signal |
| Patient's channel | No signal (no head movement) |
| Image channel | Ox = k random(n) |
| | Oy = 1 random(n) |
| | Where random is a generator of random numbers |
| | triggered at every programmable time interval “t”. |

This random foveal stimulus presented in Table 2 stimulates the saccadic system. The object changes its position every ‘t’ seconds (programmable ‘t’). The saccadic system is a position servo system through which objects within the visual field can be voluntarily placed on the fovea. It is used to define faces, reading, etc. Its stimulus-response latency ranges from about 150 to 200 milliseconds.

The cerebral cortex has a representation of this system at the level of the frontal and occipital lobes. The co-ordination of horizontal saccadic movements is similar to that of the smooth pursuit eye movement at the protuberance (gaze pontine substance), and co-ordination for vertical plane movements at the brain stem in the pretectal area. It has cerebellar afferents responsible of pulse-tone co-ordination at the level of the oculomotor neurons. The training of this conjugate oculomotor command improves retinal image stability through pulse-tone repetitive stimulation on the neural networks involved.

TABLE 3
Optokinetic reflex

| | |
|---|---|
| Auditory channel | No signal |
| Patient's channel | No signal (no head movement) |
| Image channel | An infinite sequence of objects is generated that |
| | move through the display at a speed that can be |
| | programmed by the operator. |

The retinal stimulus indicated in Table 3 trains the Optokinetic reflex. It is called retinal stimulus because it is generated on the whole retina, thus triggering an involuntary reflex. The Optokinetic reflex is one of the most relevant to retinal image stabilization strategies and one of the most archaic from the phylogenic viewpoint. This reflex has many representations in the cerebral cortex and a motor co-ordination area in the brain stem.

To trigger this reflex the system generates a succession of images moving in the direction previously set by the technician in the stimulus generating system 180. The perceptual keys (visual flow direction and velocity, and object size and color) are changed to evaluate the behavioral response of the patient to stimuli. These stimuli are generated on the display of the virtual reality goggles 152 and the patient may receive this visual stimulation while in a standing position and also while walking in place.

As this Optokinetic stimulus is permanently experienced by a subject during his/her daily activities, for example, while looking at the traffic on the street, or looking outside while traveling in a car, it can be generated by changing the perceptual keys that trigger the Optokinetic reflex. These perceptual keys are received by the patient in a static situation i.e., in a standing position, and in a dynamic situation i.e., while walking in place. This reproduces real life situations, where this kind of visual stimulation is received.

The rotation angle of the patient walking in place in the direction of the visual flow, which is normal, or in the opposite or a random direction, will progressively mark various characteristics of postural response and of normal or pathologic gait to this kind of visual stimulation.

TABLE 4
Vestibulo-oculomotor reflex

| | |
|---|---|
| Auditory channel | Programmable frequency tone “F”. |
| Patient's channel | The patient moves the head horizontally matching end |
| | positions with the tone. When the patient is capable of |
| | making a soft movement this may be represented as: |
| | Cx = k cos Π F t, where F is the tone frequency in the |
| | auditory channel. |
| Image channel | Ox = −Cx (with a display screen, Ox = 0) |

This stimulus of Table 4 trains the vestibulo-oculomotor reflex. The patient moves the head fixing the image of a stationary object on the fovea. The coordinates of the real object do not change, as the algorithm computes the patient's movement detected by the accelerometer, and shows the image after compensating the movement of the head in full.

This allows stimulation of the angular velocity accelerometers located in the crests of the inner-ear semicircular canals. Movement of the patient along the x or y plane, or along a combination of both at random, will generate oculomotor responses that will make the eyes move opposite in phase to the head in order that the subject may be capable of stabilizing the image on the retina when the head moves. According to the algorithm, the VRU system 100 senses, through an accelerometer 155 attached to the virtual reality helmet 150, the characteristics of the patients head movements (axis, direction and velocity) and generates a stimulus that moves with similar characteristics but opposite in phase. For this reason, the patient perceives the static stimulus at the center of his/her visual field

The VRU program generates symbols (letters and/or numbers) on this stimuli that change periodically and that the patient must recognize and name aloud. This accomplishes two purposes.

First, that the technician controlling the development of the rehabilitation session may verify that the patient is generating the vestibulo-oculomotor reflex that enables him/her to recognize the symbol inside the object. This is especially determining in elderly patients with impaired concentration.

Second, to test the patient's evolution. In numerous circumstances the patient has a deficit of the vestibulo-oculomotor reflex and finds it difficult to recognize the symbols inside the object. In the course of the sessions devoted to vestibulo-ocular reflex training, icon recognition performance begins to improve.

When the subject achieves the compensation of the vestibulo-oculomotor reflex, the percentage of icon recognition is normal. Visual and vestibular sensory information are “fused” in this stimulus to train a reflex relevant to retinal image stabilization

TABLE 5
Suppression of the vestibulo-oculomotor reflex

| | |
|---|---|
| Auditory | Programmable frequency tone “F”. |
| channel | |
| Patient's | The patient moves the head horizontally matching end |
| channel | positions with the tone. When the patient is capable of making |
| | a soft movement this may be represented as: |
| | Cx = k cosΠFt, where F is the tone frequency in the auditory |
| | channel. |
| Image | Ox = 0 (with a display screen, Ox = Cx) |
| channel | |

Table 5 indicates the stimulus that trains the suppression of the vestibulo-oculomotor reflex. The patient moves the head fixing on the fovea the image of an object accompanying the head movement. This stimulation reproduces the perceptual situation where the visual object moves in the same direction and at the same speed as the head. For this reason, if the vestibulo-ocular reflex is performed, the subject loses reference to the object.

In this situation the vestibulo-oculomotor reflex is “cancelled” by the stimulation of neural networks inhibiting the cerebellum (Purkinje strand) and inhibits the ocular movements opposite in phase to the head movements placing the eye ball “to accompany” head movements. This inhibition is altered in some cerebellar diseases, and the successive exposure to this perceptual situation stimulates post-lesion compensation and adaptation

TABLE 6
Vestibulo-optokinetic reflex

| | |
|---|---|
| Auditory | Programmable frequency tone “F”. |
| channel | |
| Patient's | The patient moves the head horizontally matching end |
| channel | |
| | positions with the tone. When the patient is capable of |
| | making a soft movement this may be represented as: |
| | Cx = k cos Π F t, where F is the tone frequency in the |
| | auditory channel. |
| Image | An infinite sequence of objects is generated that move |
| channel | through the “real” visual field at a speed that can be |
| | programmed by the operator. |
| | When the patient moves in the same direction, he/she tries to |
| | “fix” the image on the retina. This reflex is stimulated by the |
| | generation of a movement on the display as follows: |
| | Velocity (Ox) = programmed velocity − velocity (head) |
| | (with a display screen, Velocity (Ox) = programmed |
| | velocity) |

This stimulus of Table 6 trains the vestibulo-optokinetic reflex. When the patient “follows” the object, its movement on the display slows down. When it moves in the opposite direction, its movement on the displays becomes faster. This type of stimulation has been designed to generate a simultaneous multisensory stimulation in the patient, the perceptual characteristics of which (velocity, direction, etc., of the stimuli) should be measurable and programmable.

The patient must move the head in the plane where the stimulus is generated, and the visual perceptual characteristic received by the patient is modified according to the algorithm. This reproduces real life phenomena, for example, an individual looking at the traffic on a street (optokinetic stimulation) rotates his/her head (vestibular stimulation), and generates an adaptation of the reflex (visual-vestibular reflexes) in order to establish retinal image stability.

In patients showing damage to the sensory receptors or to the neural networks of integration of sensory information the reflex adaptation of this “addition” of sensory information is performed incorrectly and generates instability. The systematic exposure to this visual and vestibular stimulation through different perceptual keys stimulates post-lesion adaptation mechanisms.

This combined stimulation (vestibular and visual) is also generated in the patients through changes in somatosensory information, alteration of the feet support surface (firm floor, synthetic foam of various consistencies). This is a real life sensory probability where the subject may obtain visual-vestibular information standing on surfaces of variable firmness (concrete, grass, sand). This wide spectrum of combined sensory information aims at developing in the patient (who is supported by a safety harness) postural and gait adaptation phenomena in the light of complex situations where sensory information is multiple, for example, an individual going up an escalator or walking in an open space such as a mall, rotating his/her head and at the same time looking at the traffic flow from a long distance, e.g. 100 m. The software generates this “function fusion” to generate combined and simultaneous stimuli of variable complexity and measurable perceptual keys.

The VRU 100 also has a remote mode that enables it to work remotely from the patient over a network, for example, but not limited to, the World Wide Web, a Local Area Network (LAN) and a Wide Area Network (WAN). In these cases, the VRU 100 includes a register of users 116 that permits it to identify those people that it is treating and in this way only changes data pertinent to them and their corresponding training sessions. A web mode may also enable the VRU to work with a patient while information of the patient is stored remotely on another VRU or a server or other device accessible via the internet. Such remotely-stored information may include personal information regarding the patient, patient history, records of previous sessions, protocols and exercises.

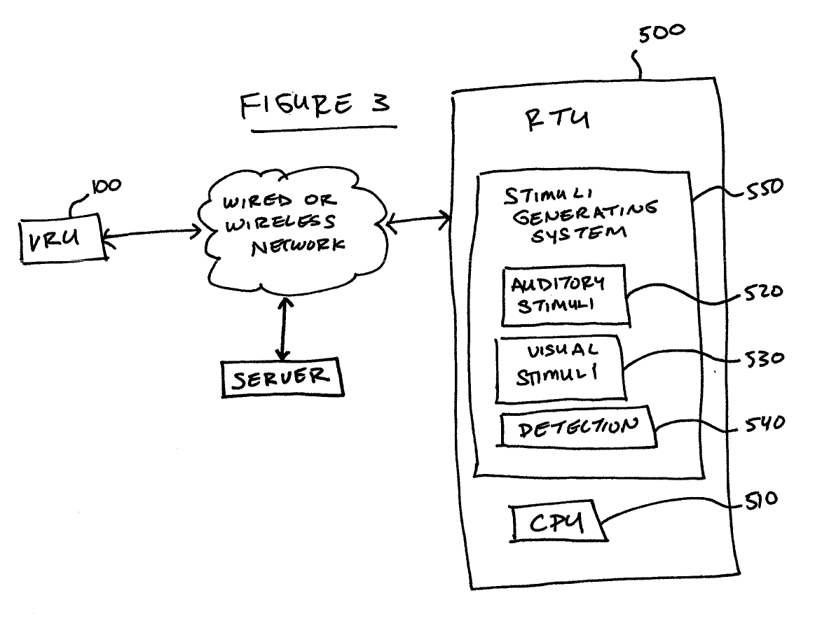

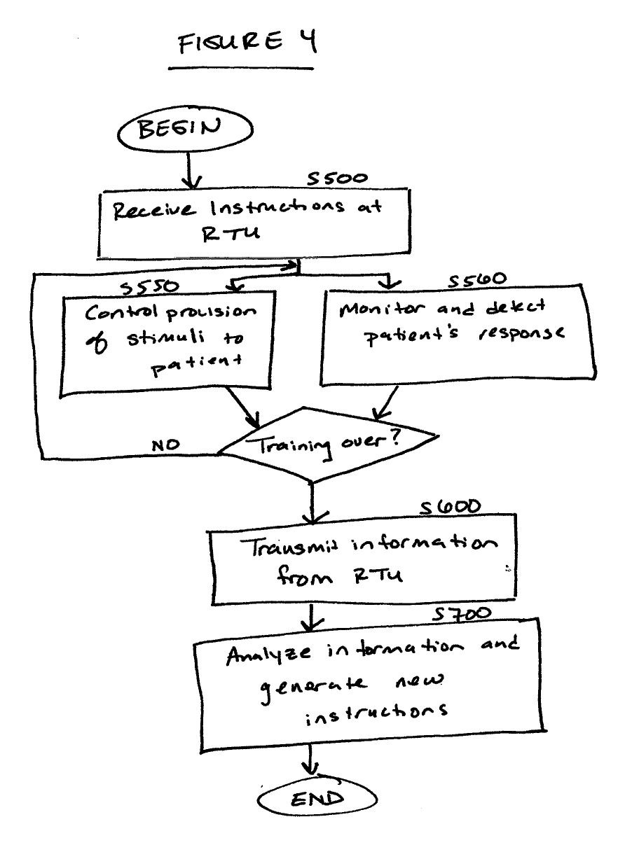

Thus, according to another exemplary embodiment, the above-described systems and methods may be applied to a system and method which enables patients to receive treatment in their homes or at another remote location. The remote system may be installed in the home of a patient or other remote location, or may be a portable device. FIGS. 3 and 4 illustrate an exemplary system and method according to this embodiment.

According to this embodiment, a remote training unit (RTU) 500 is located at the remote location. This, as shown in FIG. 3, includes a stimulus generating system and a CPU.

The stimulus generating system of the RTU 500 may include some or all of the features and elements of the VRU, as described above. Alternately, certain features may be duplicated on the VRU and RTU. A very simple version of the RTU may reproduce video files, and may be custom-made or may comprise a commercially-available device such as an mp4 player. The PRTU may also simply comprise a device capable of accessing information via the internet to provide stimulation via a display screen, which may also operate in conjunction with peripherals, such as a camera. A complex version of an RTU may be capable of not only reproducing video files, but also include posturography (PST) and Videonystagmography (VNG) functionality, discussed in more detail below. Thus, the RTU may operation according to instructions which can be downloaded or uploaded onto the RTU.

According to this embodiment, the stimulus generating system 550 may include a means for providing visual and auditory stimuli and a means for simultaneously detecting vestibulo-ocular and vestibule-spinal responses of a user in response to the stimuli 540. Based on the instructions, the CPU controls the stimulus generating system 550 to provide stimuli to the user (S550). The stimulus generating system 550 may include one or more of a visual stimuli module 530, an auditory stimuli module 520, a head posture detection module, a somatosensorial stimuli module, a virtual reality helmet, and related accessories, such as goggles, earphones, a head tracker, a display screen, and a television. The RTU 500 may also include a personal computer, a laptop or notebook computer, a commercial game console, portable multimedia system or mp4 player, or other means of providing stimulus to the patient.

The stimulus generating system 550 operates as described above, controlled by a CPU 510 based on software instructions. The CPU 510 controls the stimulus generating system 550 as described above with respect to previous embodiments.

An exemplary embodiment of a method of the present invention is illustrated in FIG. 4. According to this exemplary embodiment, the software instructions are received at the RTU (at CPU 510) via a computer-readable medium, or from a remote VRU 100 or server via a cable, wire, or fireless transmission to the RTU 500 (S500). Thus, the RTU may interact with a remotely-located VRU or a server or other device accessible via the internet, to obtain information. This permits a remote clinician or therapist to design instructions for sessions specific to a user and to provide the instructions to the RTU 500 so that the user may perform the rehabilitation and therapy sessions remotely from a specialized clinic. The instructions may be provided to the RTU 500 as software, such as pre-programmed instructions on an SD card or the like.

As described above with respect to previous embodiments of the VRU, stimuli are provided to the patient (S550) and, simultaneously, information regarding the user's responses to the stimuli are detected (S560). This information may include vestibule-ocular and vestibule-spinal responses of the user including measurements of movements of a user's head and eyes, and measurements of a user's center of gravity (COG) and body center of pressure (COP).

Such measurements may be made by accessories including a web camera, which may be mounted to virtual reality goggles, and a force platform. Ocular movements may be recorded by means of a camera, video, web-cam, or other device that can be mounted on the virtual reality goggles or mounted in another location, such as in a case in which a television is used as a screen. Vestibule-spinal information such as COP is recorded by means of a force platform, and COG movements may be recorded by optical means. Head tracking may be performed by an accelerometer or other means, such as graphical means using a camera to record the head location and detect its position. The body center of gravity (COG) is a function of the positions and masses of the particles that comprise the body. For many purposes, the body's mass behaves as if it were concentrated at the COG. as the body moves, the COG moves. A center of pressure (COP), which is the result of the forces applied by the feet, may be measured. For example, the COP varies with the distribution of weight on the feet. When a subject moves his COG, in order to maintain an upright position, some shifting must occur which produces a measurable change in the pressures exerted by the feet on the ground. The subject's COG maybe estimated based on a measured COP.

When the COP is measured and sampled, the result is a cloud of points that correspond to the COP trajectory. An area of the cloud may be estimated with an ellipse of confidence. According to this method, statistical methods are used to approximate an ellipse to the sampling of points.

The body SV is the speed at which the COP is moving.

Thus, the RTU 500 may have functionality of both recording ocular movements through imaging (Videonystagmography (VNG) functionality) and recording and utilizing COP measurements using a force platform or the like (posturography (PST) functionality). Alternately, as noted above, some of this functionality may be shared by the VRU.

PST: The main concept of PST is the recording of a body's COP by means of a force platform. The area of the COP and sway velocity are parameters for body postural control assessment. These parameters may be detected by the detection unit 540.

The VRU and/or RTU may be improved by the addition of a force platform capable of performing PST. This force platform may be a part of the detection unit 540. The system can simultaneously provide visual stimulation while recording a center of pressure of a patient standing on the platform. This improvement is aimed at detecting the postural response that a patient has to each type of stimulation viewed under a concept of control systems. The body is regarded as a system which may be considered to have three main inputs: visual, vestibular, and somatosensorial. The brain processes the information from those sources to give an output, which commands an ocular-motor and a spinal response. The spinal response may be measured by means of the PST. The parameters that can be obtained with the PST may provide a comprehensive assessment and a means to design patient rehabilitation.

VNG: Two main outputs of the balance system are the ocular-motor and spinal responses. The ocular-motor response is involved in stabilizing the image on the retina. As part of the evaluation of the balance system as a control system, the ocular-motor reflexes can be measured by means of the VNG. The VNG functionality may also be embodied in the detection unit 540.

The VRU and/or RTU may be improved by the addition of this device for recording eye movements. the system can simultaneously provide the visual stimulation while recording eye movements generated by the visual stimulation. This improvement may enable detection of the ocular response that a patient has to each type of stimulation, and the parameters that can be obtained with the VNG may also provide a comprehensive assessment and a means to design patient rehabilitation.

The VNG and PST measurements may be recorded and processed simultaneously and synchronously (S560). Thus, there may be a simultaneous analysis of the vestibulo-ocular and vestibulo-spinal functions, as well as head movements.

The information detected during use of the RTU 500 may be transmitted, via a computer-readable medium, or a cable, wire, or wireless transfer, to the VRU location or other location of a clinician or therapist for analysis and feedback (S600). The detected information may also be recorded on memory at the RTU or remotely at the VRU or other location. Among other information, the RTU may also record and transmit information regarding a session log sequence, parameters of a session, and if and when training is interrupted or restarted.

The information detected during use of the RTU 500 may then be used to generate new instructions (S700). This processing of the information and generation of new instructions may be performed at the RTU, or remotely at the VRU or other device to which the information has been transmitted.

The remote use of the RTU 500 provides virtual reality technology and environmental sensory stimulation to a patient without requiring the patient to travel to a clinic. Of course, the patient may still need to visit a clinic for further assessment, particularly to measure the patients progress, and to perform additional rehabilitation. However, in conjunction with rehabilitation in the clinic, a therapist can prepare a rehabilitation program for the patient to perform remotely with the RTU 500.

In accordance with this embodiment, the RTU 500 is able to reproduce the stimulation offered by the VRU. Information of the usage of the RTU 500 is provided to the VRU 100 (S600) and together with a new assessment (S700), a therapist can update a patient's rehabilitation program and update the information provided to the RTU 500 to deliver new stimulation sessions (S800).

In accordance with exemplary embodiments of the invention, a system is able to perform an assessment or functional diagnosis of the balance system of a patient by stimulating the patient and measuring various responses. The stimulation may be visual, auditory, somatosensorial, or motion detected by the vestibules. Measurements may be made with the PST and VNG functionality. This means that with sensory stimulus generated by virtual reality technology, it is possible to access the kind of failure of vestibular and postoral functions in patients with balance disorders. The performance of the patient may be obtained while the patient is being stimulated under different paradigms, and an analysis may determined which stimulation provided a greater challenge to the balance control system. Thus, those specific reflexes and sensory integrations can be targeted during rehabilitation.

Generally, environmental sensory input involved in the balance system may be reproduced while measurements are taken in real time of the postural and ocular responses, in order to establish the characteristic of the sensory input which impairs the postural control in patients with balance disorders.

The VRU/RTU may be used to assess a functional state of a patient's balance system and detect which type of stimulation or combination of stimuli challenges the patient the most. this information can then be used to develop a target rehabilitation program in which the patient is exposed to stimuli that recreates those challenges in order to unleash the plasticity mechanism of the brain. Following the principles of Vestibular Rehabilitation, the patient is stimulated successively with the sensory information which triggers the balance disorder. Sensory nervous system adaptation can then be tracked. The stimulation may be delivered to the patient via the VRU or the RTU.

It should be emphasized that the above-described embodiments of the present invention are merely possible examples of implementations, merely set forth for a clear understanding of the principles of the invention. Many variations and modifications may be made to the above-described exemplary embodiments of the invention without departing substantially from the spirit and principles of the invention. All such modifications and variations are intended to be included herein within the scope of this disclosure and the present invention and protected by the following claims.

What is claimed is:

1. A vestibular rehabilitation method comprising:
receiving instructions at a remote training unit (RTU);
controlling a stimulus generating system of the RTU to provide stimuli to a user based on the instructions;
while the stimuli is being provided to the user, simultaneously detecting information regarding the user's response to the stimuli from the RTU;
recording the detected information.

2. The vestibular rehabilitation method as recited in claim 1, wherein the receiving instructions comprises uploading software instructions to the RTU.

3. The vestibular rehabilitation method as recited in claim 1, wherein the receiving instructions comprises wirelessly transmitting instructions, from a vestibular rehabilitation unit (VRU) located at central location, to the RTU.

4. The vestibular rehabilitation method as recited in claim 1, wherein the receiving instructions comprises downloading instructions via the internet.

5. The vestibular rehabilitation method as recited in claim 1, wherein controlling the stimulus generating system comprises providing visual and auditory stimuli to the user.

6. The vestibular rehabilitation method as recited in claim 5, wherein detecting the information regarding the user's response comprises detecting at least one of eye movements of the user, movement of a center of pressure of the user, and a sway velocity of the user.

7. The vestibular rehabilitation method as recited in claim 5, wherein detecting the information regarding the user's response comprises simultaneously detecting vestibulo-ocular and vestibulo-spinal body responses of the user.

8. The vestibular rehabilitation method as recited in claim 7, further comprising:
transmitting the stored information from the RTU to a vestibular rehabilitation unit (VRU) located at a central location;
creating modified instructions at the central location based on the transmitted information; and
transmitting the modified instructions from the central location to the RTU.

9. The vestibular rehabilitation method as recited in claim 7, further comprising:
transmitting the stored information from the RTU to a central location via the internet.

10. The vestibular rehabilitation method as recited in claim 9, wherein transmitting the stored information from the RTU to the central location comprises transmitting the stored information from the RTU to a web server at the central location.

11. The vestibular rehabilitation method as recited in claim 10, further comprising:
creating modified instructions at the web server based on the transmitted information; and
transmitting the modified instructions from the web server to the RTU.

12. The vestibular rehabilitation method as recited in claim 7, further comprising
creating modified instructions based on the stored information; and
controlling the stimulus generating system based on the modified instructions.

13. The vestibular rehabilitation method as recited in claim 5, wherein providing visual stimuli to the user comprises providing visual stimuli via virtual reality goggles.

14. The vestibular rehabilitation method as recited in claim 5, wherein providing visual stimuli to the user comprises providing visual stimuli via a display screen.

15. A vestibular assessment method comprising:
providing instructions to a training unit;
controlling a stimulus generating system of the training unit to provide stimuli to a user based on the instructions;
while providing the stimuli, simultaneously detecting responses of the user comprising one or more of a center of pressure (COP) of the user, a sway velocity (SV) of the user, and eye movements of the user; and
analyzing the detected responses and designing a rehabilitation program based on the detected responses.

16. A vestibular rehabilitation method comprising:
transmitting instructions from a central unit to a remote training unit (RTU);
controlling a stimulus generating system of the RTU, based on the instructions, thus providing visual and auditory stimuli to a user;
while providing the stimuli to the user, simultaneously detecting vestibulo-ocular and vestibulo-spinal body responses of the user; and
transmitting inform tion regarding the detected vestibulo-ocular and vestibulo-spinal body responses of the user from the RTU to the central unit.

17. The vestibular rehabilitation method as recited in claim 16, wherein the central unit is a vestibular rehabilitation unit (VRU).

18. The vestibular rehabilitation method as recited in claim 17, wherein vestibule-ocular and vestibule-spinal body responses of the user comprise a center of pressure of the user, a sway velocity of the user, and eye movements of the user.

19. A Remote Training Unit, comprising:
a means for exchanging information with a central unit;
a means for storing instructions;
a stimulus generating means for providing stimulus to a user based on the instructions;
a means for detecting information regarding the user's response to the stimulus, simultaneously with the providing the stimulus to the user;
means for recording the detected information.

20. The Remote Training Unit as recited in claim 19 wherein:
the means for communicating with the central comprises means for communicating via the internet.

21. The Remote Training Unit as recited in claim 19, further comprising:
means for modifying the stimulus provided by the stimulus generating means, based on the detected information.

22. The Remote Training Unit as recited in claim 19, wherein the stimulus generating means comprises one of virtual reality goggles and a display screen.

23. The Remote Training Unit as recited in claim 19, wherein the information regarding user's response to the stimulus comprises a center pf pressure and sway velocity of the user.

24. The Remote Training Unit as recited in claim 19, wherein the central unit comprises a Vestibular Rehabilitation Unit (VRU).

25. The Remote Training Unit as recited in claim 20, wherein the central unit comprises a web server.

26. The Remote Training Unit as recited in claim 18, wherein the information regarding the user's response to the stimulus comprises eye movements of the user.
